(19) United States
(12) Patent Application Publication
(10) Pub. No.: US 2012/0207269 A1
(43) Pub. Date: Aug. 16, 2012

(54) X-RAY GENERATING DEVICE WITH ELECTRON SCATTERING ELEMENT AND X-RAY SYSTEM

(75) Inventor: Rolf Karl Otto Behling, Norderstedt (DE)

(73) Assignee: KONINKLIJKE PHILIPS ELECTRONICS N.V., Eindhoven (NL)

(21) Appl. No.: 13/501,464

(22) PCT Filed: Oct. 21, 2010
(86) PCT No.: PCT/IB2010/054765

§ 371 Date: Apr. 12, 2012

(30) Foreign Application Priority Data
Oct. 28, 2009 (EP) 09174310.4

(51) Int. Cl. H01J 35/04; G01N 23/04; H01J 35/30; A61B 6/03; B82Y 99/00

(52) U.S. Cl. 378/4; 378/121; 378/137; 378/62; 977/742; 977/949

(57) ABSTRACT

The present invention relates to X-ray generating technology in general. Providing X-ray generating device internal voltage sources or potentials may help reduce necessary feed-throughs into an evacuated envelope of an X-ray generating device. Consequently, an X-ray generating device comprising an electron scattering element is presented. According to the present invention, an X-ray generating device is provided, comprising an electron emitting element 16, an electron collecting element 20 and an electron scattering element 42. A primary electron beam 17a is arrangeable between the electron emitting element 16 and the electron collecting element 20. The electron emitting element 16 and the electron collecting element 20 are operatively coupled for generating X-radiation 14.

### TECHNICAL FIELD OF THE INVENTION

The present patent application relates to X-radiation generating technology in general. More particularly, the present patent application relates to an X-ray generating device, an X-ray system and the use of an X-ray generating device in at least one of an X-ray system and a CT system. In particular, the present invention relates to an X-ray generating device having an electron scattering element.

### BACKGROUND OF THE INVENTION

An X-ray system having an X-ray generating device, e.g. an X-ray tube, may generate electromagnetic radiation for acquiring X-ray images in e.g. medical imaging applications, inspection imaging applications or securing imaging applications.

An X-ray generating device regularly comprises an electron emitting element, e.g. a cathode element, and an electron collecting element, e.g. an anode element. An electron beam is formed between the electron emitting element and the electron collecting element by accelerating electrons between the electron emitting element and the electron collecting element by a potential or voltage difference.

Electrons of the electron beam impinge on an area of the electron collecting element, so constituting a focal spot or focal track, thereby generating electromagnetic radiation or X-radiation by electron bombardment of, e.g. a disk element, in particular a rotating disk element, of the electron collecting element.

One application of an X-ray generating device is in a computed tomography system or CT system. The X-ray generating device is situated on a gantry with an X-ray detector arranged on the opposite side of the gantry. Both the X-ray generating device and the X-ray detector are rotatable about an object, e.g. a patient, while the X-ray generating device is generating a fan-beam of X-rays.

X-radiation passes through the object arranged in the path between the X-ray generating device and the X-ray detector, is attenuated by the internal structure, density distribution or tissue distribution of the object and subsequently arrives at the X-ray detector. The detector converts X-radiation to electrical signals for subsequent reconstruction and display of an X-ray image of an object's inner structure by a computer system.

The acceleration of electrons from the electron emitting element to the electron collecting element regularly takes place within an evacuated envelope or housing of the X-ray generating device. For various functions of the X-ray generating device, external connections, e.g. for supplying a high voltage or heating current, have to be provided between the outside of the X-ray generating device, in particular the outside of the evacuated housing and the inside of the same. Since an according connection has to be able to withstand increased stress and has to provide a reliable connection while maintaining the vacuum of the housing, an according connection requires a highly robust implementation, which may significantly contribute to manufacturing costs of an X-ray generating device.

For various internal functions of an X-ray generating device, various feed-throughs may be required for providing multiple connections from a high voltage generator as well as connections for controlling and heating.

It may be beneficial to be able to provide, e.g. current source elements or elements being able to influence a potential, within an X-ray generating device without the need to specifically provide a high voltage feed for each element by an external feed-through.

### SUMMARY OF THE INVENTION

There may be a need to provide an X-ray generating device with the capability of generating an individual high voltage, current or potential within the evacuated housing on demand without providing a dedicated feed-through for each conceivable source element. Thus, there may be a need to provide an X-ray generating device with an electron scattering element.

In the following, an X-ray generating device, an X-ray system and the use of an X-ray generating device in at least one of an X-ray system and a CT system according to the independent claims are provided.

According to an exemplary embodiment of the present invention, an X-ray generating device is provided, comprising an electron emitting element, an electron collecting element and electron scattering element. A primary electron beam is arrangeable between the electron emitting element and the electron collecting element, wherein the electron emitting element and the electron collecting element are operatively coupled for generating X-radiation.

According to a further exemplary embodiment of the present invention, an X-ray system is provided, comprising an X-ray generating device according to the present invention and an X-ray detector. An object is arrangeable between the X-ray generating device and the X-ray detector with the X-ray generating device and the X-ray detector being operatively coupled such that an X-ray image of the object is obtainable.

According to a further exemplary embodiment of the present invention, an X-ray generating device according to the present invention is used in at least one of an X-ray system and a CT system.

One aspect of the present invention may be seen as providing a potential between an electron emitting element and a grid element or aperture element for focusing and/or switching an electron beam, in particular switching the electron beam on and off.

For fast switching of an electron beam, e.g. switching the electron beam on and off without actually disconnecting a high voltage generator, an auxiliary electrode element or grid element may be employed.

The auxiliary electrode element may be placed in the vicinity of the electron emitting element of an X-ray generating device. The grid element and the electron emitting element may have a voltage difference, thus a potential arranged between the two elements, by which potential the electron beam may be influenced. E.g., by a suitable potential between the electron emitting element and the grid element, the electron beam may be switched on and off. In other words, the voltage difference may provide for an electrical field strong enough for repelling electrons emanating from the electron emitting element, thus prohibiting the electrons to arrive at the electron collecting element.

The minimal absolute value of the potential between the electron emitting element and the grid element that prohibits the formation of an electron beam is called the cut-off voltage.

The electrical field between the electron emitting element and the electron collecting element may have a so-called penetration factor. E.g., the higher the potential between the electron emitting element and the electron collecting element, the higher the penetration factor or through-grip of the accelerating electric field may be. With a rising penetration factor, the cut-off voltage may rise accordingly. The electron emitting element with a large penetration factor may require a large cut-off voltage. However, in case the cut-off voltage is provided, e.g. by dedicated cabling, charging capacities of the cabling may be slow and energy-consuming.

The electron emission, e.g. the electron beam from the surface of a thermoionic emitter, e.g. an electron emitting element, may be depending on the pulling electrical field generated by the electron collecting element, in particular being arranged between the electron emitting element and the electron collecting element.

For fast switching, the auxiliary electrode element is placed in the vicinity of the emitter. An according auxiliary electrode element has been e.g. also employed in radio tubes, there being constituted by a grid of wires. Thus, even in a current X-ray generating devices the auxiliary electrode element may be referred to a grid element despite the fact that is may not resemble an actual wire grid.

A preferred implementation of the grid element is as an aperture element, which may be part of the electrostatic focusing of the electron emitting element cap or cathode cup. For shutting off an electron beam, a voltage may be applied to the grid element to generate a repelling electrical field.

Thus, a resulting electrical field occurs at the surface of the electron emitting element, which may be the sum of the electrical field generated by the grid element to the electron emitting element and the electron collecting element to the electron emitting element. In case the sum of both overlaying fields is repelling, in particular on all locations of the electron emitting element, electron emission is substantially completely cut off.

The minimal absolute value for prohibiting electron emission for obtaining an according potential between the electron emitting element and the grid element is referred to as cut-off voltage. The cut-off voltage may be proportional to the voltage of the X-ray generating device or X-ray tube voltage, which may be seen as the difference between the electron emitting element and the electron collecting element potential.

To accelerate switching of the grid element and to reduce a capacity to be recharged, the main voltage for the grid element may be generated inside the X-ray generating device, in particular the cathode cup of an X-ray tube only, by employing an electron scattering element, e.g. a scattered electron amplifier. The cable capacity of an external high voltage generator may be decoupled from the electron emitting element, e.g. by a divider transformer, thus obtaining an electrically floating electron emitting element or main electron emitter of the X-ray generating device. Consequently, only a reduced capacity may have to be recharged.

A relatively small external control voltage, e.g. about 1 kV, may be fed through the high voltage cable to the cathode cup for controlling an emission of an auxiliary electron emitting element for providing an auxiliary electron beam. The auxiliary electron emitting element may be connected to the negative high voltage of an external high voltage generator. However, since the control voltage may be considered to be low, a charging time due to the cable capacitance may be considered to be quite fast despite of the stray capacity of the cable.

In the following, the control voltage may be considered to be amplified by the electron scattering element. The electron current of the auxiliary electron emitting element, thus the auxiliary electron beam, may pass an auxiliary control grid and is subsequently directed towards and impinging on an electron scattering element or a scatter electrode, which may be connected to the primary electron emitting element of the X-ray generating device. With a substantially flat angle of incidence of the electrons of the auxiliary electron beam on the electron scattering element and suitable surface conditions, e.g. ceramics or an oxide coating, possibly comprising high-z-material and further a finned or whiskered structure of the electron scattering element, the scatter coefficient, i.e. the number of released electrons per incident electron, may be >1. In other words, the electron scattering element releases more electrons than it receives by the auxiliary electron beam.

The electrons emitted by the electron scattering element may be collected e.g. by a metal housing of the X-ray generating device, e.g. the X-ray tube frame. In case the electron scattering element is conductively coupled with the primary electron emitting element, the main emitter may charge positive with respect to the grid element, which may be connected to the negative high voltage supply of the external high voltage generator. The main emitter may charge positive due to the electrically floating arrangement employing a divider transformer.

The voltage difference or potential between the grid element or aperture and the electron emitting element may be limited, e.g. by a Zener diode, in particular an external Zener diode or even within the X-ray generating device by a field emitting surface of the grid element, which may e.g. be coated with carbon nano tubes. Similar to the behaviour of a Zener diode, an emission current of a field emitter may rise steeply with an applied electrical field. Thus, a suitable, well-defined cut-off voltage may be generated.

Accordingly, fast grid switching even with a high emissivity electron emitting element may be obtainable. The control voltage may be considered to be small compared to the tube voltage provided by an external high voltage generator. Thus, the reliability of high voltage cables and plugs may be improved. A further connection between the outside and the interior of an X-ray generating device may be avoided, possibly reducing manufacturing costs.

A further aspect of the present invention may be seen as providing radial electrostatic deflection of an electron beam, thus the focal spot, on e.g. a rotating disk element of the electron collecting element. Regularly, the electron beam of a diagnostic X-ray tube may comprise a rectangular shape, whereby the radial dimension may be substantially longer compared to the tangential dimension. An according radial electrostatic deflection of the electron beam may require a strong electric field, which may be generated by a voltage in the order of magnitude of the X-ray tube voltage itself, possibly allowing for a wide deflection gap while avoiding disturbances then the intensity profile of the electron beam. Generating and supplying an according high voltage from outside the X-ray generating device into the interior may be tedious task, possibly requiring a dedicated connection through the housing into the evacuated envelope of the X-ray generating device.

Accordingly, an electron scattering element or a scattered electron amplifier may be employed for generating and controlling a deflection potential inside the evacuated housing. In particular, the electrostatic beam deflection may be controlled by a relatively small control voltage, as compared to the tube voltage, which may be close to ground potential.

An auxiliary electron emitting element may provide an auxiliary electron beam impinging on an electron scattering element. As described earlier, having a scattering coefficient >1, an electrode of at least two electrode elements, between which the electron beam is to be deflected, may be charged positive, compared to the further deflection electrode element. Accordingly, an electrical field is generated between the deflection electrode elements, possibly influencing the path of electrons travelling between an electron emitting element and an electron collecting element in between the two deflection electrode elements.

An auxiliary electron emitting element may provide an auxiliary electron beam to a scatter surface of an electron scattering element, possibly providing an electron backscatter coefficient >1. The current of impinging electrons of the auxiliary electron beam may be controlled by a potential of the auxiliary electron emitting element with respect to a further acceleration element, e.g. a grounded shielding cup.

The electron scattering element may be electrically conductive connected to one deflection electrode element, which deflection electrode element may be additionally grounded by a shunt resistor. The shunt resistor may serve to reduce the sensitivity of the electrode potential with respect to small changes of the electrode charge and may therefore stabilize the electrode potential.

By controlling the emission current of the auxiliary electron beam, the auxiliary electron emitting element may control charging and discharging of the scatter surface of the electron scattering element and thus of the deflection electrode element it is connected to. Thus, the potential between the at least two deflection electrode elements through which the primary electron beam is travelling or being deflected, may vary between substantially ground potential, e.g. +1 kV, up to substantially positive high voltage level, e.g. Uₚₕᵥₗ−1 kV. Accordingly, by varying the deflection potential, i.e. the potential between the at least two deflection electrode elements, the position of the focal spot of the primary electron beam on the electron collecting element may be varied.

Instead of deflecting a single auxiliary electron beam, two different auxiliary beams may be used as well, the intensity of which may be individually controlled by steering grid structures as described before.

It may also be conceivable to not only provide one electron scattering element to one deflection electrode element but to connect to each deflection electron element a dedicated electron scattering element, for controlling the electron beam both radially inward and radially outward.

Further, it may be conceivable to control multiple deflection electrode elements by a combination of an electron scattering element and an auxiliary electron emitting element. An auxiliary electron emitting element comprising its own auxiliary deflection electrode elements for steering and/or deflecting the auxiliary electron beam may vary a potential by impinging on multiple electron scattering elements subsequently or even possibly at the same time.

So, an electrostatic beam deflection may be achievable that is controlled by a relatively small voltage close to ground potential.

According to a further aspect of the present invention, an ion collector may be provided within the evacuated housing of the X-ray generating device. An according ion collector may be provided as an auxiliary electrode element having a negative bias. The negative bias may be supplied externally by a feed-through into the evacuated envelope of the X-ray generating device from the outside or may be generated by self-charging employing an electron scattering element. A potential may be limited by an external Zener diode, possibly requiring a vacuum feed-through.

The negative potential of an ion collecting element or an ion collecting electrode element may be generated, e.g. by a self-charging by collimated scattered electrons or collimated electrons of the primary electron beam. E.g., a slotted disk element, possibly a rotating disk element of an electron collecting element, may comprise slots that allow electrons of a primary electron beam to pass beyond the surface of an electron collecting element, the focal spot and an electron aperture element.

A collimated electron beam, e.g. the part of the primary electron beam passing through the slots of the slotted disk element, may be directed under grazing incidence onto the surface of an electron scattering element having a high backscatter coefficient η>1. The scatter surface of the electron scattering element may be opposed to a pull electrode, e.g. the electron collecting element, possibly connected to ground potential. The scatter surface may be connected to the ion collector.

The angle of incidence onto the scatter surface of the primary electron beam, the part of the primary electron beam passing through the slots of the slotted disk element, may be depending on the potential of the electron scattering element and a possibly prevailing repelling electrical field. The more the electron beam is repelled by a increasing negative potential, the flatter the angle of incidence may become, thus further increasing the backscatter coefficient η. The charging of the ion collector may stop in case the net charge transfer, i.e. electrons impinging on the electron scattering element minus backscattered current is substantially zero for a certain potential.

Thus, no separate feed-through and no external supply may be required for negatively biasing the ion collector. Utilizing electron scatter, the slope of the function net current to voltage

$$\frac{d {I}_{net}}{d U}$$

of the present invention may be increasingly steeper than in case of a simple electron collector with attached shunt resistor to ground and therefore the potential of the ion collector may be considered to be much better defined.

It is also conceivable to provide the scatter surface as a curved surface and to vary the distance between the scatter surface versus the pull electrode opposing the scatter surface of the ion collector. Both measures may improve the

$$\frac{d I}{d U}$$

slope.

In the following, further embodiments of the present invention are described referring in particular to an X-ray generating device. However, these explanations also apply to the X-ray system and the use of an X-ray generating device in at least one of an X-ray system and a CT system.

It is noted that arbitrary variations and interchanges of single or multiple features between claims and in particular between claimed entities, are conceivable and within the scope and disclosure of the present patent application.

According to a further exemplary embodiment of the present invention, the electron scattering element may have a scatter coefficient >1. The electron scattering element may be a scattered electrode amplifying element.

An according electron scattering element may be adapted to release more electrons than it receives. In other words, one electron impinging on the electron scattering element may provide the release of at least one, e.g. 2-10 electrons, being released from the electron scattering element. Accordingly, an electron scattering element may be charged positive by impingement with electrons.

According to a further exemplary embodiment of the present invention, the X-ray generating device may further comprise an auxiliary electron emitting element, wherein the auxiliary electron emitting element may be adapted to provide an auxiliary electron beam for impingement on the electron scattering element.

So, e.g. by steering or controlling the auxiliary electron beam, the amount of electrons impinging on the electron scattering element may be influenced as well. Accordingly, charging or discharging of the electron scattering element may be controlled by the auxiliary electron beam and thus the auxiliary electron emitting element.

According to a further exemplary embodiment of the present invention, the electron scattering element may be adapted to positively charge and/or negatively charge an element of the X-ray generating device.

In particular, the electron scattering element may positively charge an element to which it is in conductive connection, while a further element may be negatively charged by impingement of scattered electrons.

According to a further exemplary embodiment of the present invention, the electron scattering element may be adapted to influence a potential of the X-ray generating device.

E.g., by positively charging an element of the X-ray generating device, the element may behave as if a voltage would have been applied to the element. Accordingly, since a potential may also be understood as a difference in applied voltages, the electron scattering element may influence a potential between an element, e.g. to which it is connected to and a further element, having a relatively fixed voltage, e.g. connected to ground or negative high voltage of the high voltage generator.

According to a further exemplary embodiment of the present invention the X-ray generating device may further comprise a grid element, wherein the electron scattering element may be adapted to influence a potential of the grid element for providing a cut-off voltage to the X-ray generating device.

The potential of the grid element may in particular be understood as a potential between the electron emitting element and the grid element. By providing an according potential to the grid element, an electrical field may be provided possibly counteracting the electrical field employed for accelerating electrons of the primary electron beam between the electron emitting element and the electron collecting element, thus repelling electrons and so hindering the electrons from being emitted by the electron emitting element for subsequent acceleration towards the electron collecting element.

According to a further exemplary embodiment of the present invention, the X-ray generating device may further comprise a deflection element, wherein the electron scattering element may be adapted to influence an electrical field of the deflection element and wherein the primary electron beam may be deflectable by the deflection element.

Thus, by influencing a potential of a deflected element, in particular between two deflection electrode elements, an electrical field arranged in between the deflection electrode elements may be influenced. By influencing an according electrical field, the path of electrons passing through the electrical field may be influenced as well, possibly providing a relocation of the focal spot on the electron collecting element.

According to a further exemplary embodiment of the present invention, the X-ray generating device may further comprise an ion collecting element, wherein the electron scattering element may be adapted to influence one of a potential and a bias of the ion collecting element.

By providing either a positive charge or negative charge to an element within the evacuated envelope of the X-ray generating device, an ion collecting element may be generated, adapted to collect either positively charged or negatively charged ions.

According to a further exemplary embodiment of the present invention, the electron scattering element may be one element out of the group consisting of a surface, a surface element, a moderator element, a finned element, a whiskered element, a wire grid, an element comprising one of a dynode coating, beryllium oxide (BeO), aluminum oxide (Al₂O₃), magnesium oxide (MgO), salt of the formula xCl, xBr, metal surfaces comprising metallic elements uranium (U), niobium (Nb), tungsten (W), tantalum (Ta), molybdenum (Mo), rhenium (Rh), titanium (Ti), Diamond crystals, doped Diamond crystals, Diamond foil, doped Diamond foil, Carbon Nano Tubes and Fullerenes.

In particular employing an element having a finned or a whiskered structure possibly further comprising a coating may allow to further increase the scatter coefficient from >1 up to 2-10.

These and other aspects of the present invention will become apparent from and elucidated with reference to the embodiments described hereinafter.

Exemplary embodiments of the present invention will be described below with reference to the following drawings.

The illustration in the drawings is schematic. In different drawings, similar or identical elements are provided with similar or identical reference numerals.

Figures are not drawn to scale, however may depict qualitative proportions.

### BRIEF DESCRIPTION OF THE DRAWINGS

FIG. 1 shows an exemplary embodiment of an X-ray system according to the present invention;

FIG. 2 shows an exemplary embodiment of an X-ray generating device comprising a control grid;

FIG. 3 shows an exemplary embodiment for providing a cut-off voltage employing an electron scattering element according to the present invention;

FIG. 4 shows a first embodiment for deflecting an electron beam comprising an electron scattering element according to the present invention;

FIG. 5 shows a second embodiment for deflecting an electron beam comprising an electron scattering element according to the present invention;

FIG. 6 shows an exemplary embodiment of providing an ion collection element comprising an electron scattering element according to the present invention;

FIGS. 7A-D show individual implementations of a rotating disk element of an electron collecting element having slots according to the present invention;

FIGS. 8-9C show exemplary embodiments of electron backscattering; and

FIGS. 10A-C show exemplary electron backscatter coefficient values according to the present invention.

### DETAILED DESCRIPTION OF EMBODIMENTS

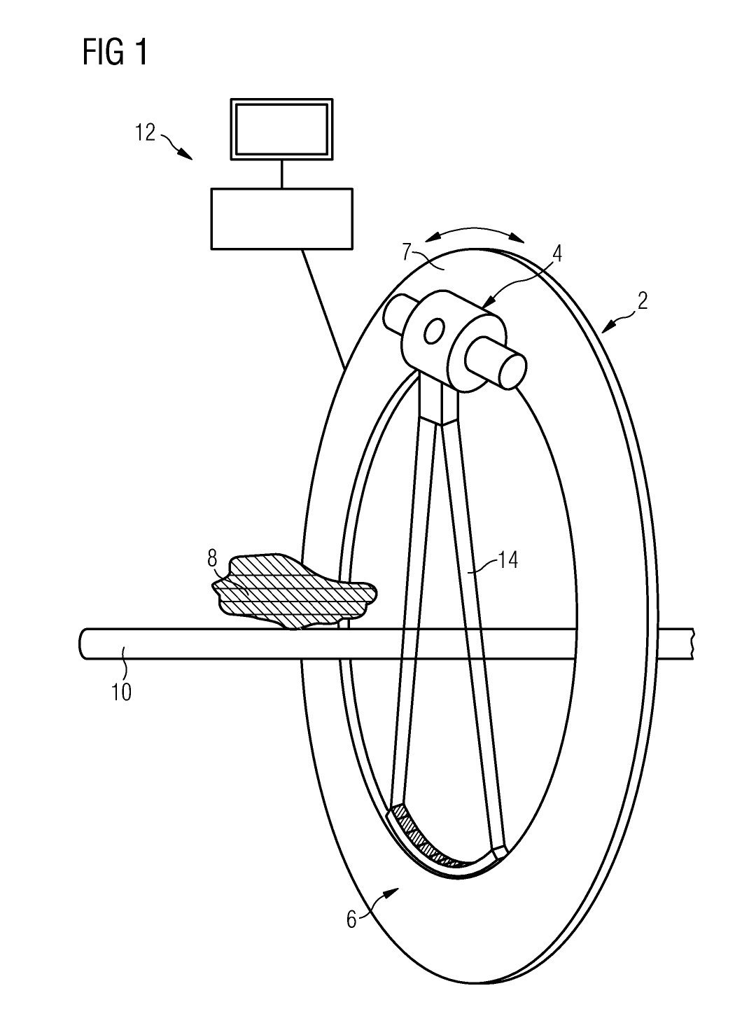

Now referring to FIG. 1, an exemplary embodiment of an X-ray system according to the present invention is depicted.

In FIG. 1, an X-ray system 2 comprising an X-ray generating device 4 and an X-ray detector 6 is presented. X-ray generating device 4 and the X-ray detector 6 are arranged on a gantry 7 opposing one another for rotation about an object 8 arranged on a support 10. The X-ray generating device is emanating X-radiation 14 in the direction of the X-ray detector 6, possibly penetrating object 8, which subsequently spatially attenuates the X-ray fan-beam 14 before impinging on X-ray detector 6. A control system 12 is provided for controlling the acquisition of X-ray images by the X-ray system 2 and for displaying and calculating X-ray images of the acquired data by the X-ray detector 6.

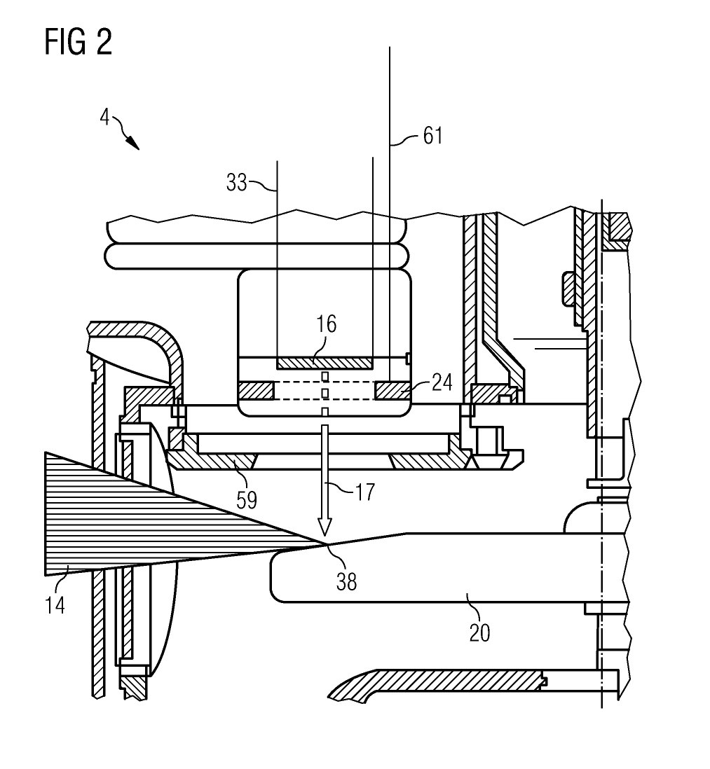

Now referring to FIG. 2, an exemplary embodiment of an X-ray generating device comprising a control grid is depicted.

In the FIG. 2, a detailed view of a cut-through of an X-ray generating device 4, e.g. an X-ray tube, is depicted. X-ray generating device 4 is exemplary an X-ray generating device 4 having an electron collecting element 20 implemented as a rotating anode element. Electron emitting element 16 is generating an electron beam 17 towards the electron collecting element 20. The electrons of the electron beam 17 impinging on the electron collecting element 20 generate X-radiation 14. The electron beam is impinging on the focal spot 38 of the electron collecting element 20.

An aperture element 24 is provided in the vicinity of electron emitting element 16, being provided with a grid voltage 61 for controlling, e.g. switching on and off electron beam 17. An electron aperture element 59 is provided for directing and/or focusing the electron beam on the electron collecting element 20.

By grid voltage 61 supplied to aperture element 24, an electrical field between aperture element 24 and electron emitting element 16 may be generated, possibly repelling emitted electrons, thus hindering them from forming an electron beam 17.

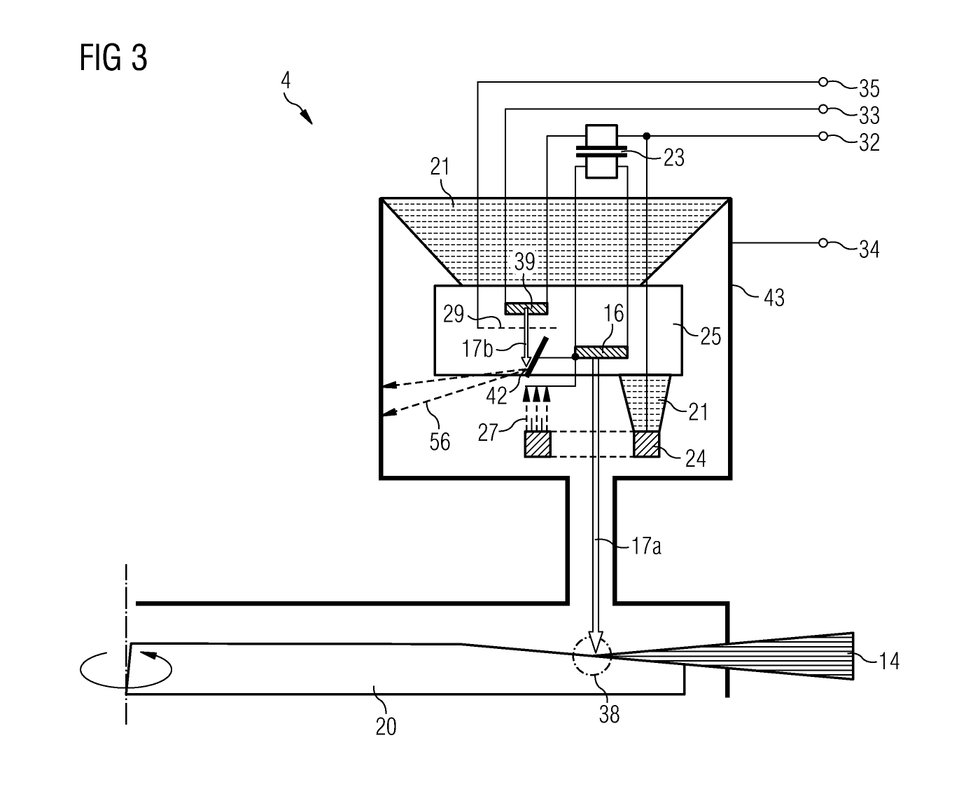

Now referring to FIG. 3, an exemplary embodiment for providing a cut-off voltage employing an electron scattering element according to the present invention is depicted.

X-ray generating device 4 comprises an electron emitting element 16, which is generating a primary electron beam 17a towards electron collecting element 20, in particular focal spot 38.

The housing 43 of X-ray generating device 4 is connected to ground potential 34. An aperture element 24 or grid element 24 for switching the electron beam 17a on and off is provided. Grid element 24 is directly connected to the negative voltage supply without a dedicated control voltage.

By employing divider transformer 23, the electron emitting element 16 is electrically floating. Connected to electron emitting element 16 is electron scattering element 42, which is connected electrically conductive.

A heating current feed-through 33 is provided for both an auxiliary electron emitting element 39 and the electron emitting element 16. The auxiliary electron emitting element 39 is arranged to provide an auxiliary electron beam 17b towards the electron scattering element 42. The auxiliary electron beam 17b is controlled by auxiliary control grid 29, which is connected to control voltage feed-through 35.

Employing the control voltage 35, the auxiliary electron beam 17b may be controlled, i.e. switched on and off and its current set. The electrons of the auxiliary electron beam 17b impinge on the electron scattering element 42 under a flat angle of incidence, possibly generating backscatter electrons 56, which are impinging on the grounded housing 43 of the X-ray generating device 4.

The electron scattering element 42 has a backscatter coefficient larger than 1. In other words, for a single electron of the electron beam 17b impinging on the electron scattering element 42, at least one, e.g. 2-10, backscatter electrons 56 are released.

Accordingly, electron scattering element 42 is charged positive due to loosing more electrons than it is receiving. Due to the electrically conductive connection to electron emitting element 16 and with the electrically floating arrangement of electron emitting element 16, the floating potential of the electron emitting element 16 is raised also and thus a potential difference between the grid element 24 and the electron emitting element 16 occurs, resulting in an electrical field 27.

The electrical field 27 is thus counteracting the acceleration electrical field between the electron emitting element 16 and the electron collecting element 20. In case the electrical field 27 is completely repelling electrons from the electron emitting element 16, the formation of an electron beam 17a is prohibited.

Accordingly, providing an according electrical field 27 may result in switching on an off the primary electron beam 17a. Thus, by control voltage 35, the auxiliary electron beam 17b may be controlled, by which impingement on the electron scattering element 42 the primary electron beam 17a and thus the generation of X-radiation 14 may be controlled.

The field 27 between the grid element 24 and the electron scattering element 42 may be seen as a field emission voltage limiter, since the current rises exponentially with a potential difference.

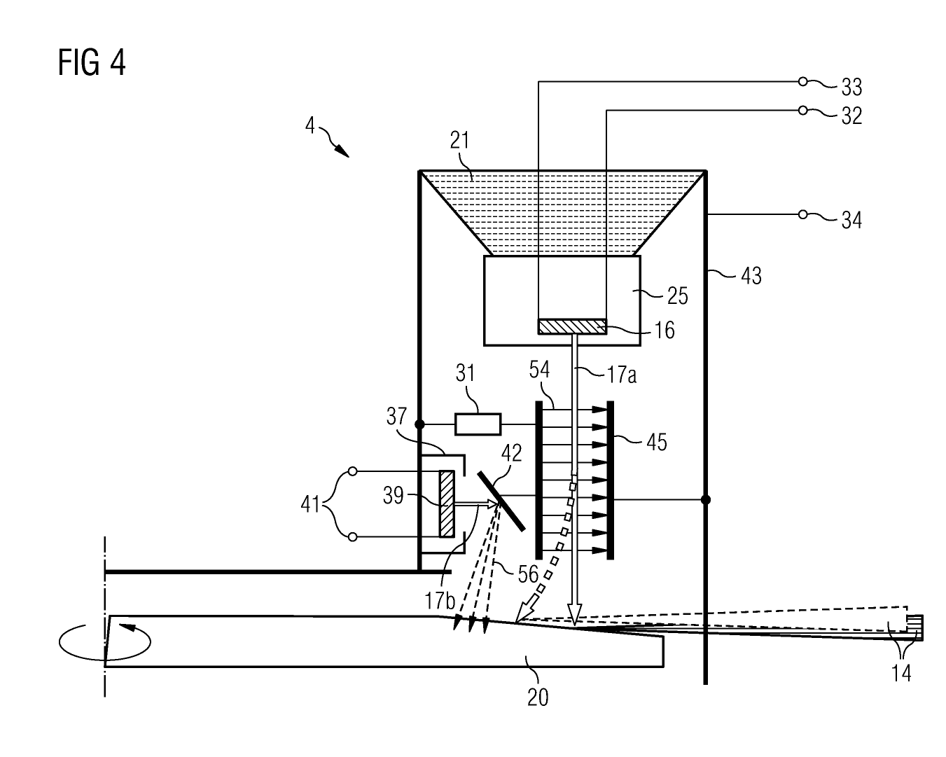

Now referring to FIG. 4, a first embodiment for deflecting an electron beam comprising an electron scattering element according to the present invention is depicted.

X-ray generating device 4 comprises an electron emitting element 16 for generating a primary electron beam 17a between the electron emitting element 16 and the electron collecting element 20 for generating X-radiation 14. A heating current 33 and a negative voltage 32, e.g. −120 kV, are supplied to the electron emitting element 16, possibly requiring a feed-through into the evacuated envelope or housing 43 of X-ray generating device 4. The housing 43 is connected to ground potential 34.

A support structure 25, comprising an insulator 21, is also arranged within the housing 43. Deflection elements 45 are arranged such that the primary electron beam 17a is passing between individual deflection electrode elements 45 before impinging on the electron collecting element 21. One deflection electrode element 45 is connected to ground potential 34 of the housing 43 with the other deflection electrode element 45 is connected to the housing by a shunt resistor 31.

An electron scattering element 42 is conductively connected to the deflection electrode element 45 that is connected to the housing via shunt resistor 31. An auxiliary electron emitting element 39 is provided with the housing, possibly having individual heating and voltage supply feed-throughs 41. A grounded shielded cup 37 is employed for generating an auxiliary electron beam 17b from the auxiliary electron emitting element 39 towards electron scattering element 42.

The auxiliary electron beam 17b impinges on the electron scattering element 42 thus creating backscatter electrons 56, which may e.g. be directed to the electron collecting element 20. As before, electron scattering element 42, due to its conductive connection to one deflection electrode element 45, is positively charging the respective deflection electrode element 45, thus creating an electrical field 54 between the deflection electrode elements 45.

By influencing the secondary electron beam 17b, thus how the electron scattering element 42 positively charges one of the two deflection electrode elements 45, the path of the primary electron beam 17a may be influenced, e.g. radially, as depicted in FIG. 4 by the slotted arrow of primary electron beam 17a.

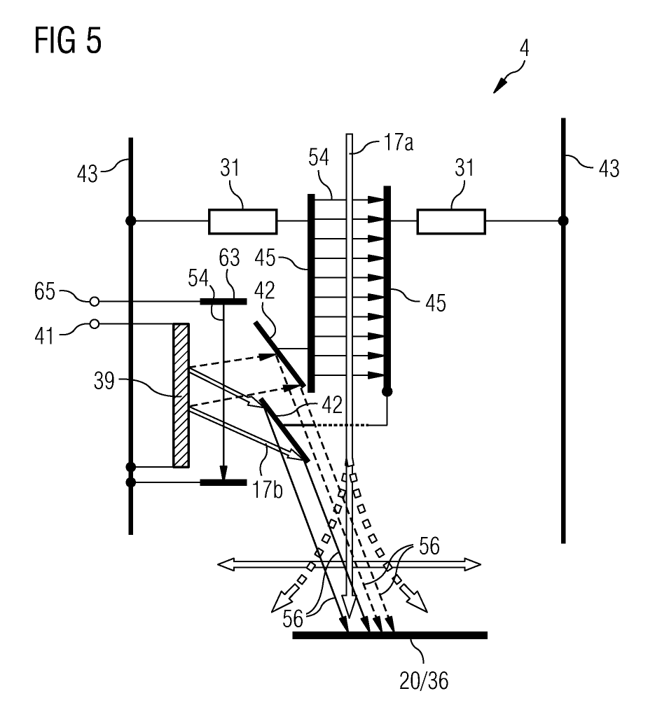

Now referring to FIG. 5, a second embodiment for deflecting an electron beam comprising an electron scattering element according to the present invention is depicted.

FIG. 5 is similar to the implementation of FIG. 4, with the main difference of two shunt resistors 31 connected between each deflection electrode element 45 and ground potential 34 of the housing 43. Furthermore, each deflection electrode element 45 is connected conductively to an individual electron scattering element 42.

The auxiliary electron emitting element 39 is provided with an auxiliary deflection element 63 having its own feed-through of an auxiliary deflection element control voltage 65. By the auxiliary deflection element, in particular by control voltage 65, the auxiliary electron beam 17b may be steered towards either electron scattering element 42, possibly in a way to smoothly transition between both elements, thus dividing the auxiliary electron beam 17b between the exemplary two electron scattering elements 42.

Accordingly, the primary electron beam 17a may both be steered radially inward and radially outward. The electron collecting element 20 may either be ground potential or positive potential 36.

The scatter surfaces of the electron scattering elements 42 of both deflection electrode elements may be alternatively charged. The control voltage 65 may control the direction of the auxiliary electron beam 17b, e.g. alternating from one to the other electron scattering element 42 and with it the amount of charging by the backscatter effect.

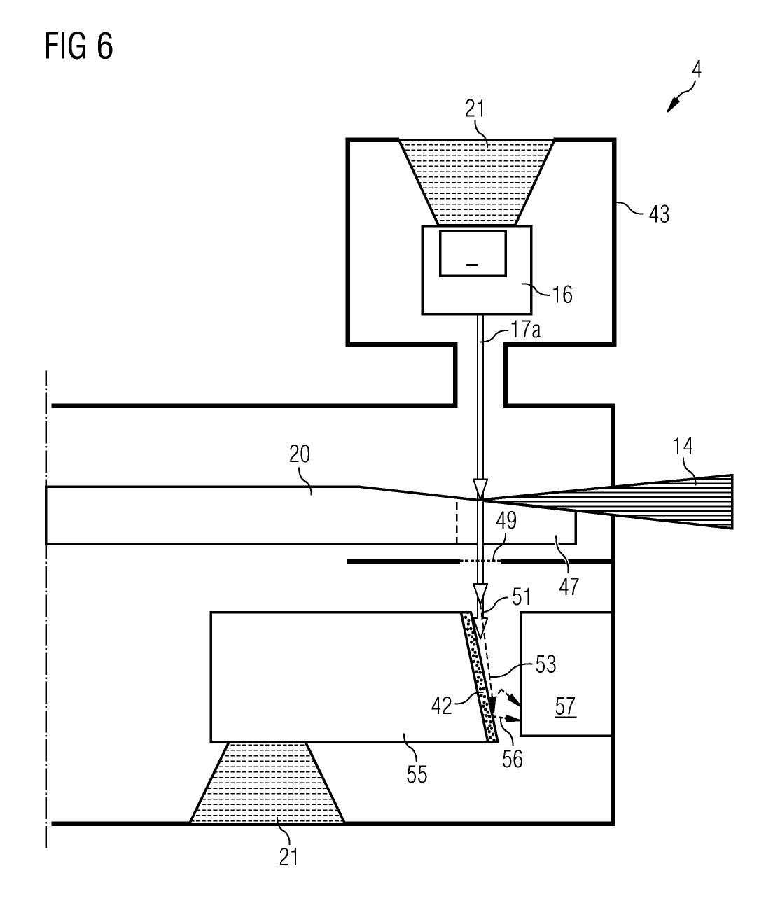

Now referring to FIG. 6, an exemplary embodiment of providing an ion collection element comprising an electron scattering element according to the present invention is depicted.

X-ray generating device 4 comprises an electron emitting element 16 arranged within evacuated housing 43, possibly attached to the housing 43 by insulator 21. Electron emitting element 16 is generating an electron beam 17a towards the electron collecting element 20 for generating X-radiation 14. The electron collecting element 20 comprises individual slots 47 that allow the passing of the electron beam 17a beyond the electron collecting element 20. An aperture element 49 is provided for further accelerating, focusing and/or directing the electron beam 17a towards the electron scattering element 42, attached to ion collector 55.

Electron beam 17a is passing the electron collecting element 20 and impinging on the electron scattering element 42 possibly generating backscattered electrons 56. A pull electrode 57 is arranged opposing the electron scattering element 42 for pulling the backscattered electrons 56 towards itself.

Accordingly, due to a scatter coefficient >1, in particular for grazing incidence, the electron scattering element 42 and thus the ion collector 54 is positively charged for collecting ions. In case the electron scattering element 42 and thus the ion collector 55 has a neutral potential, the angle of incidence is rather steeper 51 and the scatter coefficient <1, while with the electron scattering element 42 and thus the ion collector 54 having a negative potential, the angle of incidence is flattened 53, thus contributing to a further increase in the generation of backscatter electrons 56. The strong dependence of the scatter coefficient of a scatter surface on the angle of incidence may provide preferred stabilization of the potential than a simple electron collector.

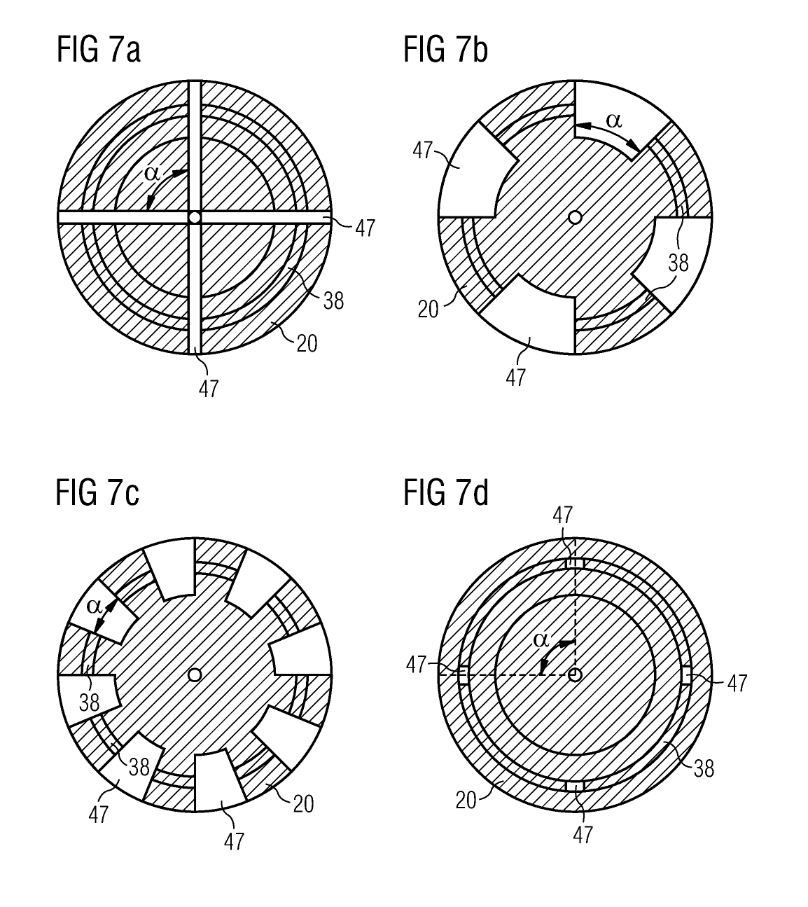

Now referring to FIGS. 7A-D, individual implementations of a rotating disk element of an electron collecting element having slots is depicted.

In FIG. 7A, slots 47 are arranged on the disk element of the electron collecting element 20 as radial slots 47 having an angle of 90° between them. Focal track 38 is indicated.

In FIG. 7B, slotted areas 47 and non-slotted areas are substantially similarly shaped and sized, having an angle of substantially 45°.

Rotating disk element according to FIG. 7C is comparable to the rotating disk element of FIG. 7A, however having twice as many slots 47, thus having an angle of 22.5° between them.

With regard to FIG. 7D, slots 47 are only cut outs in the area of a focal spot or focal track 38, in FIG. 7D exemplary 4 slots. However a different number of slots 47, e.g. 1, 2, 3, 5, 6, 7, 8, 9, 10, 11 or 12 are conceivable, possibly spaced apart employing equal angles between them.

It may be especially beneficial to have a rotatory symmetrical arrangement of slots, due to possibly high rotational speeds of a rotating disk element.

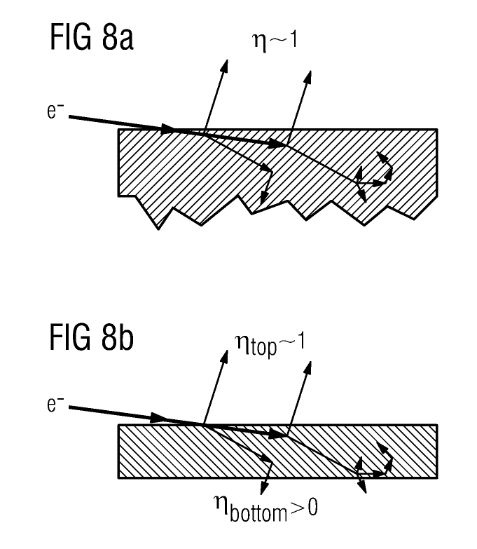

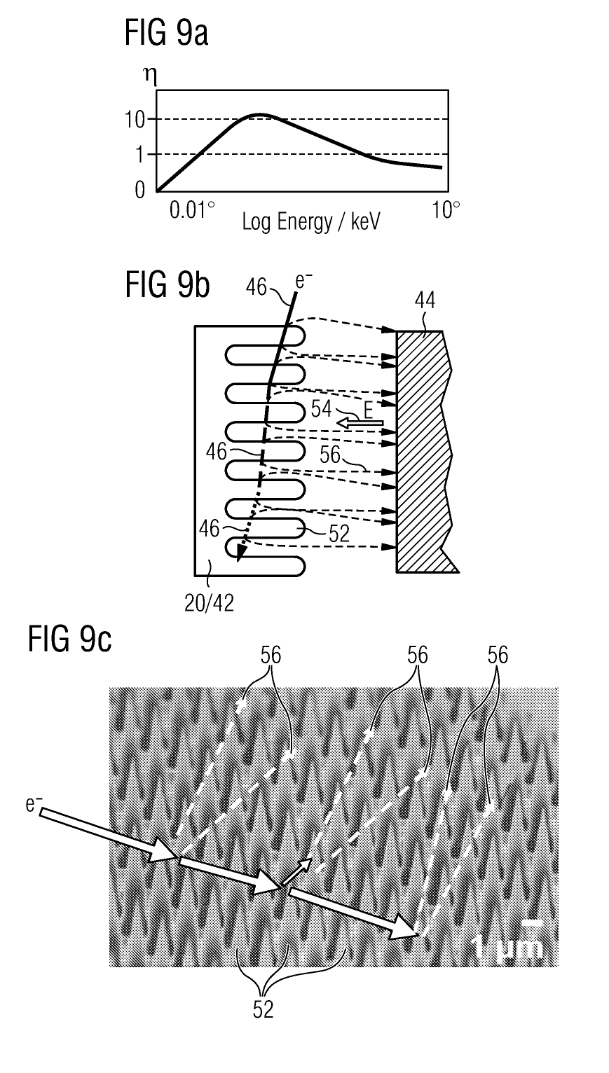

Now referring to FIGS. 8a to 9c, exemplary embodiments of electron back scattering are depicted.

In FIG. 8a, a scatter ratio η of about 1 is depicted. An electron with grazing incidence, thus a small angle of incidence, is entering into e.g. an electronically opaque surface like gold or tungsten. The electron, which is travelling within the structure, however close below the surface of e.g. a tungsten body, may interact multiply with electrons 50% of the scatter electrons may be considered to be released into the vacuum hemisphere of the X-ray generating device 4, thus constituting to about a scatter ratio of 1. The remaining 50% may get lost in the body due to multiple scattering within the body. These would at least be partly available for release as well.

With regard to FIG. 8b, in case the body of FIG. 8a may be considered to be foil or being a sort of a finned structure or whiskered structure, at least a part of the electrons otherwise lost in the body, may also be released into the vacuum, in particular on the opposing side of where the electron entered the body. This may hold in particularly true in case the thickness of the foil is within the range of the penetration depth of impinging electrons. Accordingly, a scatter ratio η>1 may be achievable by η=ηₜₒₚ+η_{bottom>}1.

With regard to FIG. 9, the back scatter ratio η is depicted vs. energy.

Dynode coatings like e.g. beryllium oxide, magnesium oxide and aluminum oxide may provide an electron scatter coefficient η of 2 to 10. Employing a sandwich structure, which employs a high-z-material like tungsten as a bottom layer, which may effectively scatter high energy electrons, and an additionally coating on top of the bottom layer with an according dynode coating or a mixture of the mentioned coating to enhanced secondary electron emission may be in particular beneficial.

With regard to FIGS. 9b, c, employing a finned structure or a whiskered structure for generating back scattered electrons 56 is depicted. The back scattering under grazing incidence may further be enhanced by a rough structure, in particular surface structure, having fins or whiskers. The protruding elements may in particular be thinner than the average penetration depth of impinging electrons 46. Thus, back scattered electrons 56 may be released from both the top side and the rear side of an individual fin, thus obtaining a scatter gain of >2, which results in a scatter ratio η>2.0, e.g. for tungsten having e.g. 80 to 150 keV.

A scatter electron 46 is entering a comb structure of the scatter element 42 having individual whiskers or fins 52. The electron, while individually penetrating multiple whiskers, is generating back scattered electrons 56, both when entering and leaving a single fin or whisker 52. The back scattered electrons 56 are accelerated by an electrical field 54 towards the scatter electron collecting element 44. Thus, a single scatter electron 46 may generate multiple back scattered electrons 56, e.g. 10, so resulting in a back scatter ratio η=10.

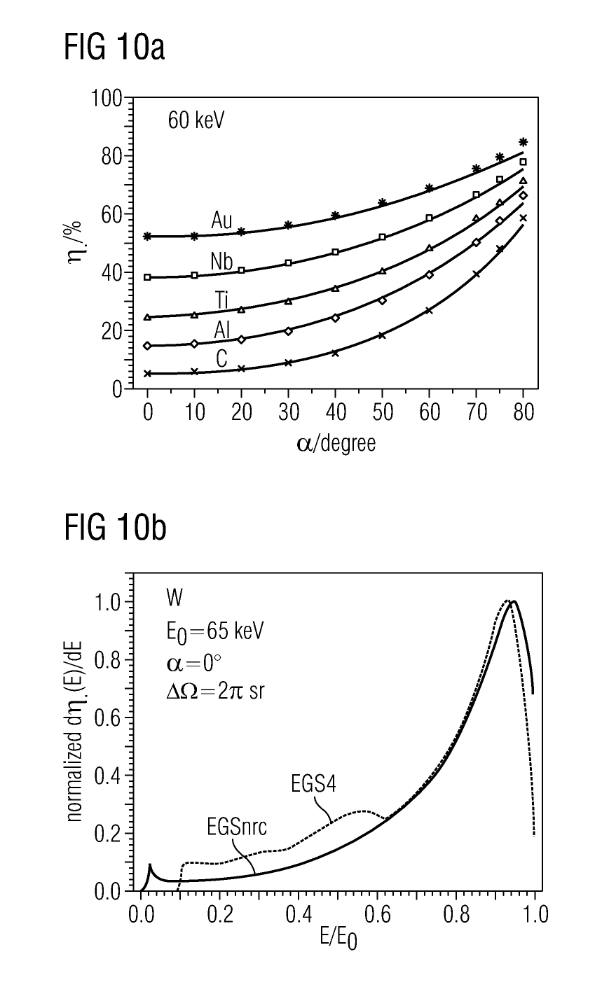

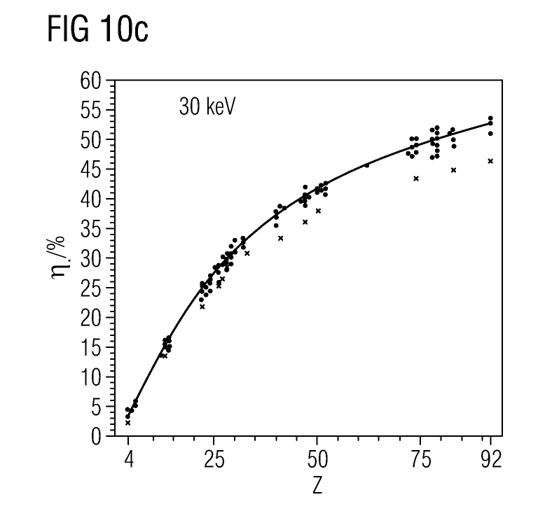

Now referring to FIGS. 10a to 10c, exemplary electron back scatter coefficient values according to the present invention is depicted.

FIGS. 10a, the electron back scatter coefficient η versus angle of incidence a for a 60 keV electron beam is depicted.

With regard to FIG. 10b, the overall energy spectrum of 65 keV electrons back scattered from a semi-infinite tungsten target is depicted. It may be taken from FIG. 10b, that despite a large number of electrons is backscattered nearly elastically, the average energy of the scattered electrons is significantly lower than the primary energy. After multiple scatter events e.g. from W-surfaces, the scattered electrons are slowed down. Such an arrangement may be used as a moderator element, which brings the average electron energy down into a range, where other materials have a high scatter yield η.

With regard to FIG. 10c, the electron back scatter coefficient η versus atomic number of a sample material Z for electrons with incident kinetic energy of 30 keV is depicted. Particularly, high-z elements provide a high scatter coefficient η and are useful as moderator elements.

It should be noted that the term “comprising” does not exclude other elements or steps and that “a” or “an” does not exclude a plurality. Also, elements described in association with different embodiments may be combined.

It should also be noted, that reference numerals in the claims shall not be construed as limiting the scope of the claims.

### REFERENCE NUMERALS

2 X-ray system
4 X-ray generating device
6 X-ray detector
7 Gantry
8 Object
10 Support
12 Control system
14 X-radiation
16 Electron emitting element
17a,b Electron beam
18 Deflection elements
20 Electron collecting element
21 Insulator
23 Divider transformer element
24 Aperture element/grid element
25 Support structure
27 Electrical field
29 Auxiliary control grid
31 Shunt resistor
32 Negative voltage
33 Heating current (feed-through)
34 Ground potential
35 Control voltage (feed-through)
36 Positive potential/voltage
37 Grounded shielded cup
38 Focal spot/focal track
39 Auxiliary electron emitting element
41 Heating and voltage supply
42 Electron scattering element
43 Housing
44 Scatter electron collecting element
45 Deflection element
46 Scatter electrons
47 Slots
49 Aperture element
51 Steeper incidence
52 Fin/whisker
53 Flatter incidence
54 Electrical field
55 Ion collecting element
56 Backscatter electrons
57 Pull electrode element
59 Electron aperture element
61 Grid voltage
63 Auxiliary deflection element
65 Auxiliary deflection element control voltage

1. X-ray generating device (4), comprising
at least one electron emitting element (16);
at least one electron collecting element (20); and
at least one electron scattering element (42);
wherein a primary electron beam is or primary electron beams are arrangeable between the electron emitting element(s) (16) and the electron collecting element(s) (20); and
wherein the electron emitting element(s) (16) and the electron collecting element(s) (20) are operatively coupled for generating X-radiation (14).

2. X-ray generating device according to claim 1,
wherein the electron scattering element(s) (42) has/have a scatter coefficient >1.

3. X-ray generating device according to claim 1,
wherein the electron scattering element(s) (42) is/are a scattered electron amplifying element(s).

4. X-ray generating device according to claim 1, further comprising
at least one auxiliary electron emitting element (39);
wherein the at least one auxiliary electron emitting element (39) is adapted provide an auxiliary electron (17b) beam for impingement on at least one electron scattering element (42).

5. X-ray generating device according to claim 1,
wherein the at least one electron scattering element (42) is adapted to positively charge and/or negatively charge at least one element of the X-ray generating device (4).

6. X-ray generating device according to claim 1,
wherein the at least one electron scattering element (42) is adapted to influence at least one potential of the X-ray generating device (4).

7. X-ray generating device according to claim 1, further comprising
at least one grid element (24);
wherein the at least one electron scattering element (42) is adapted to influence a potential of the grid element (24) for providing a cut-off voltage to the X-ray generating device (4).

8. X-ray generating device according to claim 1, further comprising
at least one deflection element (45,63);
wherein the at least one electron scattering element (42) is adapted to influence an electrical field (27,54) of the at least one deflection element (45,63),
wherein the primary electron beam (17) is deflectable by the deflection element (45,63).

9. X-ray generating device according to claim 1, further comprising
at least one ion collecting element (55);
wherein the at least one electron scattering element (42) is adapted to influence one of a potential and a bias of the ion collecting element (55).

10. X-ray generating device according to claim 1,
wherein the at least one electron scattering element (42) is one out of the group consisting of a surface, a surface element, a moderator element, a finned element, a whiskered element, a wire grid, an element comprising one of a dynode coating, beryllium oxide (BeO), aluminum oxide (Al₂O₃), magnesium oxide (MgO), salt of the formula xCl, xBr, metal surfaces comprising metallic elements uranium (U), niobium (Nb), tungsten (W), tantalum (Ta), molybdenum (Mo), rhenium (Rh), titanium (Ti), Diamond crystals, doped Diamond crystals, Diamond foil, doped Diamond foil, Carbon Nano Tubes and Fullerenes.

11. X-ray system, comprising
an X-ray generating device (4) according to claim 1; and
a X-ray detector (6);
wherein an object is arrangeable between the X-ray generating device (4) and the X-ray detector (6); and
wherein the X-ray generating device (4) and the X-ray detector (6) are operatively coupled such that an X-ray image of the object (8) is obtainable.

12. Use of an X-ray generating device according to claim 1 in at least one of an X-ray system (2) and a CT-system.
